SEQUENCE LISTING (1 sequence)

SEQ ID NO: 1 - protein, 79 aa, homo sapiens
SEAEDASLLSFMQGYMKHATKTAKDALSSVQESQVAQQARGWVTDGFSSLKDYWSTVKDKFSEFWDLDPEVRPTSAVAA

(19) United States
(12) Patent Application Publication
(10) Pub. No.: US 2004/0137521 A1
(43) Pub. Date: Jul. 15, 2004

(54) Method for apo clll measurement in apo b and non apo b containing particles

(76) Inventors: Jamila Najib-Fruchart, Santes (FR); Zouher Majd, Marcq en Barouel (FR)

Correspondence Address: NIXON & VANDERHYE, PC, 1100 N GLEBE ROAD, 8TH FLOOR, ARLINGTON, VA, 22201-4714, US

(21) Appl. No.: 10/479,756

(22) PCT Filed: Jun. 4, 2002
(86) PCT No.: PCT/EP02/06110

(30) Foreign Application Priority Data
Jun. 5, 2001 (EP) 01401445.0

(51) Int. Cl. G01N 33/53

(52) U.S. Cl. 435/7.1

(57) ABSTRACT

The present invention relates to a new method for measuring apolipoprotein CIII (“apo CIII”) in apo B and non apo B containing lipoparticles. This invention also relates to synthetic apo CIII products, corresponding antibodies, kits comprising the same, and their use to detect, quantify and/or monitor apo CIII levels in a sample, as well as to quantify and/or monitor atherogenic lipoparticle levels in a sample. The above compounds and kits can also be used to modulate apo CIII levels or activity in vitro or in vivo, and to regulate lipid metabolism in a subject.

The present invention relates to a method for the detection and quantification of apolipoprotein CIII (“apo CIII”). This invention also relates to synthetic apo CIII products, corresponding antibodies, kits comprising the same, and their use to detect, quantify and/or monitor apo CIII levels in a sample, as well as to quantify and/or monitor atherogenic lipoparticle levels in a sample. The above compounds and kits can also be used to modulate apo CIII levels or activity in vitro or in vivo, and to regulate lipid metabolism in a subject.

Increasing evidence suggest that apo CIII plays an important role in controlling plasma triglyceride metabolism and in determining plasma concentration of potentially atherogenic triglyceride-rich lipoproteins (TRL) [1]. Apo CIII, a 79 amino acids protein synthesised by the liver and the intestine [2], is a component of chylomicrons, very low density lipoproteins (VLDL) and high density lipoproteins (HDL) [3].

Apo CIII exists as three isoforms: apo CIII₀, apo CIII₁ and apo CIII₂. Apo CIII₀ is not glycosylated, however apo CIII₁, and apo CIII₂ are glycosylated and have respectively one and two sialic acid residues [4]. The sugar moiety consists of disaccharide β-D galactosyl [1-3] α-N-Acetyl-D-Galactosamine attached to threonine 74 of protein chain by O-glycosidic binding [5]. In human normolipidemic plasma apo CIII₀, apo CIII₁ and apo CIII₂ represent 14%, 59% and 27% of total apo CIII, respectively. While several variants of apo CIII are associated with moderate hyperlipidemia [6, 7], mutagenesis of the glycosylation site of human apo CIII does not affect its secretion and lipid binding [8].

Plasma concentration of apo CIII is positively correlated with levels of plasma triglycerides [9, 10]. Liver perfusion studies demonstrate that apo CIII inhibits the hepatic uptake of TRL and their remnants [11, 12], whereas in vitro experiments show that apo CIII inhibits the activity of both lipoprotein lipase (LPL) and hepatic lipase [13-17]. Apo CIII, therefore modulates the plasma catabolism and clearance of TRL. This is of pathophysiological significance as indicated by angiographic studies showing that plasma lipoprotein distribution of apo CIII is a statistically significant independent predictor of the progression or severity of coronary artery diseases (CAD) [18-20]. The role of apo CIII in plasma TRL metabolism has been more defined by the results of recent studies in transgenic animals [21]. Plasma accumulation of TRL in mice overexpressing apo CIII has been shown to be associated with reduced plasma VLDL and chylomicrons remnants clearance, apparently due to reduced binding of TRL to LDL receptor [22] and to heparan sulfate proteoglycans [23]. Also the inhibitory effect of apolipoproteins C on the LDL receptor of apo B-containing lipoproteins was demonstrated [24]. Decreased receptor binding was reversed by addition of exogenous apolipoprotein E. Immunologicals and cryo-electron microscopy studies have indicated that apo CIII masked some apo B100 epitopes and modified its conformation [23]. Furthermore, the ratio of apo CIII lipoparticles devoid of apolipoprotein B (apo CIII Lp non B) and of apo CIII lipoparticles containing apolipoprotein B (apo CIII LpB) is closely connected to the lipolytic activity and stabilization or decline of atherosclerotic plaque [25].

These data thus show that plasma levels of apo CIII can be correlated to various pathophysiological conditions involved in the atherosclerosis susceptibility and the predisposition to CAD. It is thus clearly apparent that the availability of compounds, methods and kits to detect, quantify or modulate Apo CIII levels would be of significant value in the therapeutic, diagnostic, screening and/or experimental areas.

Previous processes have been described in the art to detect apo CIII, based on immunological methods. In particular, Kashyap et al. [10] relates to a radio-immuno-assay (RIA) for measurement of apo CIII. However, the reported method is expensive and requires radioisotope handling. Holmquist [26] discloses an Enzyme-Linked Immuno-Sorbent Assay (ELISA) to quantify Apo CIII. However, this assay is a low precision test, requires antibody purification by immuno-affinity and its labelling. In addition pure apo CIII or VLDL lipoparticles are needed, when competition ELISA are used. It has also a low throughput. Curry et al. [27] reported an Electroimmunoassay method (EIA). However, large amounts of polyclonal antibodies are needed, the assay is of low throughput, and delipidation of lipemic samples may be necessary for accurate determination. It is also not as sensitive as the other methods.

In addition to the above drawbacks of prior art techniques, another important and difficult aspect of previous apo CIII immuno-assay is the preparation of antigen. Indeed, native apo CIII must be isolated from pooled human plasma. Ideal patients for such collections are patients with severe endogenous hypertriglyceridemia with fasting chylomicronemia (type V lipoprotein phenotype). Such patients are not always available, and even if the apo CIII purification is performed with this hypertriglyceridemic plasma, only few mg of this protein can be obtained. Furthermore, for the purification of native apo CIII, anhydrous solvents must be used for delipidation step because the C proteins are slightly soluble in a “wet” organic phase. This decreases recovery of apolipoproteins and invalidates any quantitative information. The procedure of delipidation can also lead to the formation of peroxide that generates artefacts in purified apolipoproteins. Another difficult aspect of the apo CIII preparation from native plasma is to obtain this protein with high purity, since the other apo C proteins (apo CI and apo CII) and apo AII have some identical physicochemical characteristics with apo CIII.

The present invention now provides a novel strategy to produce apo CIII and a new method to detect and to quantify this apolipoprotein. The present invention specifically discloses novel methods of producing efficient anti-apo CIII antibodies using total synthetic apo CIII. The invention also discloses such efficient antibodies, kits comprising the same, and their use to detect, quantify, purify and/or monitor total apo CIII levels or apo CIII in atherogenic or non atherogenic lipoparticles (LpB and Lp non B) in serum or plasma.

A particular object of this invention relates to a substantially pure, synthetic apo CIII polypeptide having the sequence of SEQ ID NO: 1, or an immunogenic fragment or derivative thereof.

A further object of this invention is a method of producing anti-apo CIII antibodies comprising an immunization step with a synthetic apo CIII polypeptide as defined above. This invention also encompasses antibodies prepared according to this method, as well as, more generally, antibodies that bind a polypeptide as defined above, as well as fragments or derivatives of such antibody.

An other aspect of this invention is a method of detecting or dosing total apo CIII or apo CIII in apo B or non apo B containing lipoparticles in plasma or serum sample with an antibody (including a fragment or derivative thereof) as defined above.

An other object of this invention is a method of detecting predisposition or individuals at risk of developing lipid-metabolism disorders, comprising detecting in vitro total apo CIII or apo CIII in apo B or non apo B containing lipoparticles in a sample from a subject with an antibody (including a fragment or derivative thereof) as defined above.

An other object of this invention is a method of monitoring hepatic uptake of triglyceride-rich lipoprotein in a subject, comprising detecting in vitro apo CIII-containing particles in a sample using an antibody (including a fragment or derivative thereof) as defined above.

An other object of this application is a method of detecting or monitoring the formation, development or progression of atherosclerosis in a subject, comprising detecting in vitro, in a sample from said subject, the level of apo CIII or apo CIII-containing atherogenic particles using an antibody (including a fragment or derivative thereof) as defined above effective. The subject is preferably a mammal, particularly a human, more preferably a subject at risk of developing lipid-disorders such as CAD or a subject having such a disease.

As indicated above, this invention disclosesa substantially pure, synthetic apo CIII polypeptide having the sequence of SEQ ID NO: 1, or an immunogenic fragment or derivative thereof. The tern “substantially pure” indicates that the polypeptide is essentially devoid of other side products that occur during apoCIII synthesis, particularly of other apolipoproteins, such as apo CI, apo CII, apo B and apo AII. The term “synthetic” indicates that the polypeptide is not a naturally-occurring molecule, but has been prepared by a synthesis using chemical processes, as described in the examples. In this regard, the synthetic polypeptide of this invention is essentially unglycosylated.

The instant invention now shows that synthetic apo CIII can be produced and used to generate antibodies. The invention further shows that such antibodies are able to specifically bind naturally-occurring apo CIII, whether as a soluble antigen or included in lipoparticles. The invention shows that such antibodies can bind to the various isoforms of apo CIII, and have immuno-precipitating properties. These synthetic polypeptides and corresponding antibodies thus represent novel advantageous products to detect and quantify apo CIII.

More particularly, the synthetic polypeptide of this invention comprises SEQ ID NO: 1, as described below:

SEQ ID NO: 1: Ser-Glu-Ala-Glu-Asp-Ala-Ser-Leu-Leu-Ser-Phe-Met-Gln-Gly-Tyr-Met-Lys-His-Ala-Thr-Lys-Thr-Ala-Lys-Asp-Ala-Leu-Ser-Ser-Val-Gln-Glu-Ser-Gln-Val-Ala-Gln-Gln-Ala-Arg-Gly-Trp-Val-Thr-Asp-Gly-Phe-Ser-Ser-Leu-Lys-Asp-Tyr-Tip-Ser-Thr-Val-Lys-Asp-Lys-Phe-Ser-Glu-Phe-Trp-Asp-Leu-Asp-Pro-Glu-Val-Arg-Pro-Thr-Ser-Ala-Val-Ala-Ala

As illustrated in the examples, this polypeptide can be prepared advantageously by solid phase synthesis, particularly using a Boc/Bzl strategy [28]. When this strategy of synthetic apo CIII is used, the production is 10 to 100 fold faster and leads to a much higher purity than prior art techniques.

The term “derivative” includes polypeptide comprising one or several mutation, substitution, deletion and/or addition of one or several amino acid residues and retaining substantially the same antigenic specificity. Typical examples of derivatives include sequence variations due to apo CIII polymorphism, splicing, etc. Most preferred derivatives contain 5 modified amino acid residues at most, as compared to SEQ ID NO: 1. Additional residues may correspond to carrier or linker residues, protecting groups, etc. Furthermore, the polypeptide may be modified, for instance by chemical, physical and/or enzymatic modification, to enhance its stability, increase its immunogenicity, incorporate a label or a tracer, etc. Examples of such modifications include addition of a tag (e.g., myc), a label (e.g., radiolabel, enzymatic label, etc.), a glycosylation, etc.

The polypeptides may be soluble, purified or complexed with a carrier molecule, such as KLH or serum-albumin, for instance, or any other inert (e.g., synthetic) molecule, including a bead, etc. In a particular embodiment, the polypeptides are coupled to a carrier, especially for use in antibody production. Coupling can be performed according to conventional techniques [29, 30].

The polypeptides may also be conjugated or fused to any heterologous polypeptide molecule, such as a biologically active molecule, for instance. Heterologous designates any polypeptide which does not originate from a human apo CIII molecule.

A specific embodiment of this invention is a composition comprising a synthetic polypeptide consisting of SEQ ID NO: 1 and devoid of other apolipoproteins.

The polypeptides may be used in screening assays, or in titration assays, as controls, standards or to calibrate the assays. They may also be used to modulate some enzymes activities (lipoprotein lipase and hepatic lipase). They are also particularly suited to produce anti-apo CIII antibodies.

In this regard, a further object of this invention resides in an antibody that binds a polypeptide as defined above. Obviously, binding should be specific, meaning that the antibody should not bind specifically to other antigens, and that the binding to other antigens can be discriminated from specific binding to the above apoCIII peptide. As illustrated in the examples, preferred antibodies of this invention do not bind specifically to distinct lipoproteins. The antibody may be a polyclonal or a monoclonal antibody. Furthermore, the term antibody also includes fragments and derivatives thereof, in particular fragments and derivatives of said monoclonal or polyclonal antibodies having substantially the same antigenic specificity. These include antibody fragments (e.g., Fab, Fab′2, CDRs, etc), humanized antibodies, poly-functional antibodies, Single Chain antibodies (ScFv), etc. These may be produced according to conventional methods, including immunization of an animal and collection of serum (polyclonal) or spleen cells (to produce hybridomas by fusion with appropriate cell lines).

Methods of producing polyclonal antibodies from various species, including mice, rodents, primates, horses, pigs, rabbits, poultry, etc. may be found, for instance, in Vaitukaitis et al. [29]. Briefly, the antigen is combined with an adjuvant (e.g., Freud's adjuvant) and administered to an animal, typically by sub-cutaneous injection. Repeated injections may be performed. Blood samples are collected and immunoglobulins or serum are separared.

Methods of producing monoclonal antibodies from various species as listed above may be found, for instance, in Harlow et al (Antibodies: A laboratory Manual, CSH Press, 1988) or in Kohler et al (Nature 256 (1975) 495), incorporated therein by reference. Briefly, these methods comprise immunizing an animal with the antigen, followed by a recovery of spleen cells which are then fused with immortalized cells, such as myeloma cells. The resulting hybridomas produce the monoclonal antibodies and can be selected by limit dilutions to isolate individual clones. Antibodies may also be produced by selection of combinatorial libraries of immunoglobulins, as disclosed for instance in Ward et al (Nature 341 (1989) 544).

Preferred antibodies of this invention are prepared by immunization with a pure synthetic apo CIII polypeptide as described above, preferably comprising SEQ ID NO: 1, or with an immunogenic sub-fragment thereof, e.g., a subfragment comprising at least an epitope.

This invention also relates to a method of producing an anti-apo CIII antibody, comprising injecting a polypeptide of SEQ ID NO: 1 or an immunogenic fragment or derivative thereof to an non-human animal and collecting the antibodies or antibody-producing cells. The method is simpler than previously disclosed methods using purified native apo CIII and allows the production of specific and immunoprecipitating antibodies. The specificity can be verified by showing the absence of cross-reactivity with other blood circulating proteins. More generally, the specificity indicates that the antibodies bind apo CIII with a higher affinity than other antigens. As illustrated in the examples, polyclonals of this invention are immunoprecipitating and can thus be used to detect or dose apo CIII with high efficacy.

The antibodies may be coupled to heterologous moieties, such as toxins, labels, drugs or other therapeutic agents, covalently or not, either directly or through the use of coupling agents or linkers. Labels include radiolabels, enzymes, fluorescent labels, magnetic particles and the like. Toxins include diphteria toxins, botulinum toxin, etc. Drugs or therapeutic agents include lymphokines, antibiotics, antisense, growth factors, etc. Methods of using such heterologous moieties are illustrated, for instance, in U.S. Pat. No. 4,277,149 and U.S. Pat. No. 3,996,345.

The antibodies of this invention have various applications, including therapeutic, diagnostic, purification, detection, prophylactic, etc.

In vitro, they can be used as screening agents or to purify the antigen from various samples, including various biological samples (e.g., blood samples). They can also be used to detect or quantify the presence (or amounts) of total apo CIII or apo CIII in apo B and non apo B containing lipoparticles in a sample collected from a subject, typically a blood sample from a mammalian, specifically a human subject.

In this regard, an other object of this invention is a method of detecting total apo CIII or apo CIII in apo B and non apo B containing lipoparticles in a biological sample, comprising contacting the sample with an antibody as defined above (including fragments or derivatives thereof) and detecting the presence of antibody-antigen immune complexes. Typically the method allows the determination of the levels of total apo CIII or apo CIII in apo B and non apo B containing lipoparticles in a sample, by assessing the (relative) amounts of immune complexes in the sample and comparing the same to a standard condition or a calibration curve, for instance. The method may be performed using any conventional technique, such as ELISA (direct or competitive immuno-assay), RIA, EIA etc. However, in a most preferred embodiment, the method is a nephelometric assay. Indeed, as indicated above, the antibodies are specific and can immunoprecipitate apo CIII in a sample.

In the nephelometric assay, the intensity of light scattered by particles in suspension is measured using an analyser. The particles are formed by the immunoprecipitation reaction that occurs in a polymer-enhancing buffer when a specific antibody is brought into contact with the specific antigen. The complexing of an antigen with an antibody specific for the antigen occurs at a rate which increases gradually at first, then rapidly, and finally proceeds through a peak value that is proportional to the antigen concentration. The assay is based on a measure of the maximum rate of change from the scattered light signal, which is correlated (and can be converted) to the antigen concentration. Typically, the nephelometric assay is performed using Beckman immunochemistry systems (IMMAGE), which presents the results on the alphametric display. The nephelometric assay of this invention is advantageous since it is rapid and reproducible and can be implemented on a high throughput basis. Indeed, this assay is performed in a few seconds only for each sample, versus one day in most prior art techniques, and the coefficient of variation is 4% only versus 10% for apo CIII detection assays described in the prior art.

A particular object of this invention thus lies in a method of measuring total apo CIII or apo CIII in apo B and non apo B containing lipoparticles in a biological sample, comprising contacting the sample (or a dilution thereof) with an antibody as described above (including fragments or derivatives thereof), typically at various dilutions thereof, and assessing the formation of apo CIII-antibody immune complexes by nephelometric assay. More preferably, the antibody is subjected to a treatment prior to being contacted with the sample, in order to remove non-immunoglobulin proteins and/or to concentrate the antibody. The treatment typically comprises contacting the antibodies with polyethylene glycol (PEG), as described for instance in Ritchie et al. (31). Typically, from 0.5 to 1 μg of specific antibodies are used in the assay, although the slilled person may use different quantities without departing from the instant invention.

In a nephelometric assay, polyclonal antibodies are generally used.

The method can be carried out on various biological samples, including plasma, serum, interstitial fluid, supernatant of cultured cells etc. The sample may be collected from a subject (e.g. a human subject) and used directly for the assay. Alternatively, the sample may be diluted and/or stocked (for instance in frozen state) for later testing.

The method can be applied to the detection of soluble apo CIII or of apo CIII-containing lipoparticles. As indicated above, apo CIII is contained in various lipoparticles such as high density lipoproteins (HDL), low density lipoproteins (LDL), very low density lipoproteins (VLDL), chylomicrons, etc. Some of these lipoparticles are atherogenic and their (relative) amounts in a sample correlate with a pathological condition of a subject. In particular, apo CIII-containing lipoparticles that further contain apolipoprotein B are known to be highly atherogenic (ref Franck Sacks, Zouher). In this respect, the ratio apo CIII LpB/apo CIII Lp non B is indicative of atherosclerosis initiation, development or progression in a subject. The present invention now allows a mass measurement of apo CIII-containing lipoparticles using anti-synthetic apo CIII antibodies, with high efficacy reliability and throughput.

More particularly, the invention can be used to detect the atherogenic, apo CIII LpB in a sample. In a particular embodiment, the method comprises the steps of:

(a) determining the amount of total apo CIII in a sample

(b) removing apo B-containing lipoparticles from the sample, and

(c) determining the amount of apo CIII in non apoB containing lipoparticles in the sample.

By deduction, the amounts obtained in (a) and (c) allow the determination of the amounts of apo CIII in apoB containing lipoparticles in the sample, said particles being the most atherogenic. Removal of apoB containing lipoparticles in step (b) can be performed by treatment of an aliquote of said sample with an immunoprecipitating anti apoB antibody.

The detection assay can be used in various experimental, clinical and/or diagnostic conditions.

In particular, the method can be used to detect predisposition of individuals at risk of developing lipid-metabolism disorders. A particular object of this invention is a method of detecting predisposition or individuals at risk of developing lipid-metabolism disorders, comprising the measurement in vitro of apo B and non apo B containing lipoparticles in a sample from a subject with an antibody as defined above (including fragments or derivatives thereof), wherein increased levels of apo CIII or apo CIII in apo B containing lipoparticles (as compared to a mean value of a normal subjects) are indicative of individuals at risk of developing lipid metabolism disorders. Typically, a significant physiopathologic apo CIII in apo B containing lipoparticles level is an increase of at least 20%, preferably at least 50% over the normal mean value.

An other object of this invention is a method of monitoring hepatic uptake of triglyceride-rich lipoproteins in a subject, comprising detecting in vitro apo CIII or apo CIII-containing lipoparticles level in a sample using an antibody as defined above(including fragments or derivatives thereof).

An other object of this invention is a method of monitoring the efficacy of a lipid-metabolism-related disorder treatment in a subject comprising detecting apo CIII or apo CIII in apo B and non apo B containing lipoparticles levels in vitro in a sample from said subject using an antibody as defined above (including fragments or derivatives thereof), after administration of said treatment to the subject. Typically, the efficacy of the treatment is correlated to the apo CIII levels in the subject. The efficacy can be correlated to the ability of the treatment to regulate apo CIII or apo CIII in apo B and non apo B containing lipoparticles level or activity or to restore normal apo CIII or apo CIII in apo B and non apo B containing lipoparticles level in a subject.

A further object of this invention is a method of evaluating the physiological state of a subject at his lipid metabolism level, comprising detecting apo CIII in apo B and apo non B containing lipoparticles levels in vitro in a sample from said subject using an antibody as defined above (including fragments or derivatives thereof).

The antibodies can also be used to screen compounds or diets that might modulate total apo CIII and apo CIII in apo B and non apo B containing lipoparticles concentration in serum. Typically, the method comprises administering a compound or subjecting an animal or patient to a diet, collecting a biological sample from the animal or patient and detecting or dosing total apo CIII and apo CIII in apo B and non apo B containing lipoparticles level in said sample using an antibody as defined above (including fragments or derivatives thereof).

As indicated above, these methods can be carried out on various samples (typically plasma or serum) and can be performed by ELISA, RIA, EIA, etc., most preferably by nephelometric assay.

This invention also relates to a kit comprising a polypeptide or an antibody as described above. The kit can be used to detect or quantify apo CIII in apo B and non apo B containing lipoparticles in any sample. Most preferred kits comprise an antibody as defined above and reagents to perform or detect (or quantify) an immune reaction, particularly an antibody-antigen complex. Reagents include labels, buffers, substrates, etc. The kits typically comprise containers for the different reagents and products, and may further comprise a support or other device suitable to perform the assay.

Further aspects and advantages of this invention will be disclosed in the following examples, which should be regarded as illustrative and not limiting the scope of this application.

### LEGEND TO THE FIGURES

FIG. 1: Antibody specificity

FIG. 2: Antibody affinity for VLDL and HDL

FIG. 3: Apo CIII nephelometric assay. Calibration curve.

FIG. 4: Correlation study

### EXAMPLES

1. Apo CIII Synthesis

The polypeptides were synthesized by the solid phase method (26) on an automated synthesizer Model ABI 431 A (Applied Biosystems Inc.) using Boc/Bzl strategy on 0.5 mmol of PAM-Ala resin. Each amino acid was coupled twice by dicyclohexylcarbodiimide/hydroxybenzotriazole without capping. The crude products were purified and analysed by reversed-phase HP-LC on a Vydac C18 column using linear gradient from 0 to 100% Buffer B (Buffer A: 0.05% TFA in H₂O and Buffer B: 0.05% TFA, 60% CH₃CN in H₂O). The molecular masses were determined using an API (Perkin-Elmer) of a simple quadrupole ion electrospray mass spectrometer equipped with an ion -spray (nebulizer-assisted electrospray) source (SCiex, Toronto, Canada). Amino acid analysis was performed using Beckman 6300 amino acid analyser (Beckman instruments, Fullerton, Calif.), after hydrolysis with 6N HCl containing 0.25% phenol at 110° C. for 24 h.

2. Immunizations

Anti-serum to apo CIII was prepared in rabbits essentially as described earlier (27). The peptide was emulsified in complete Freud's adjuvant and injected sub-cutaneously to rabbits using 0.5 mg peptide per injection for the two first injections followed at 15 day intervals with boosters in the same adjuvant but using only 0.25 mg of peptide.

3. Isolation of Anti-Apo CIII Immunoglobins (IgG)

Ig G were prepared by modified protocol of Ritchie et al (31). Non immunoglobulin proteins were removed from immune-serum and the IgG are dialysed and concentrated.

4. Specificity of the Antibodies

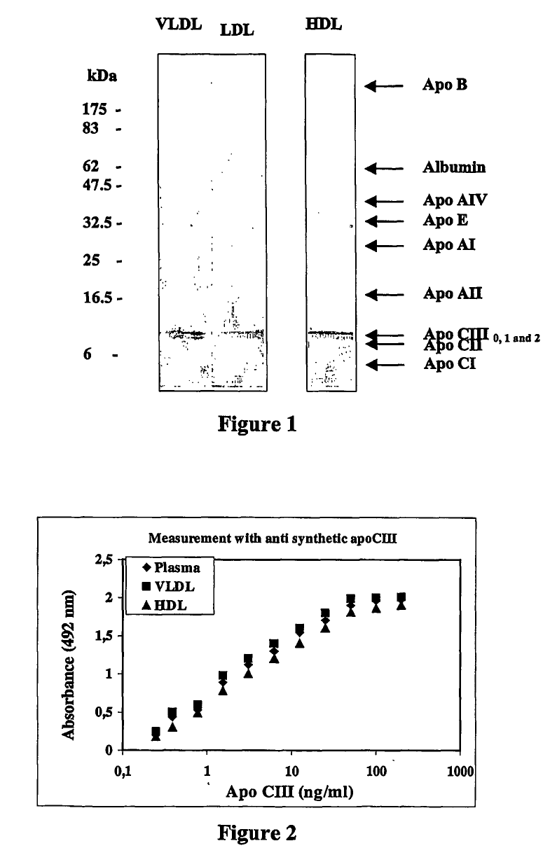

Analytical immunoblot of VLDL, LDL and HDL were performed to assess the specificity of apo CIII antibodies. As shown in FIG. 1, no cross-reaction was observed with the other proteins of all subclasses of lipoproteins, demonstrating the high specificity of the antibodies.

5. Affinity of the Antibodies Towards Apo CIII in VLDL and HDL Lipoparticles

In this experiment, we determined whether the anti-synthetic apo CIII antibodies could measure apo CIII in VLDL and HDL with the same affinity, to validate the immuno-assay in apo CIII Lp B and apo CIII Lp non B particles measurement. The results are presented in FIG. 2. The parallel curves of plasma, VLDL and HDL demonstrate the accuracy of the anti-synthetic apo CIII antibodies to recognize apo CIII in all these lipoproteins sub-classes. Furthermore, in addition to their advantageous properties and manufacturing conditions, control experiments have indicated that the present anti-synthetic apo CIII antibodies have an affinity which is at least as good as previous antibodies.

6. Immuno-Nephelometric Assay

Reagents and Materials

TABLE 1

| | design | References |
|---|---|---|
| Anti apo | 1 × 6.5 ml (300 tests) | Laboratory lot |
| CIII IgGs | | |
| Anti apo B | 1 × ml (100 tests) | Laboratory lot |
| Immunoglobulins | | |
| Diluant 1 | 4 × 120 ml (4 × 330 tests) | Beckman Coulter part No |
| | | 447640 |
| Diluant 2 | 4 × 120 ml (4 × 330 tests) | Beckman Coulter part No |
| | | 447660 |
| Buffer 1 | 4 × 120 ml (4 × 330 tests) | Beckman Coulter part No |
| | | 447650 |
| IMMAGE | 10 (10 × 300 tests) | Beckman Coulter part No |
| UDR Cartrige | | 447250 |
| Microtubes | 1000 | Beckman Coulter part No |
| | | 448162 |

Anti apo CIII Immunoglobulins:

Anti apo CIII IgG as produced in examples 2 and 3 above are ready to use. They can be refrigerated at 2 to 8° C. for use in the week, or frozen at −20° C. for use up to months. The immunoglobulins contain sodium azoture.

Anti apo B Immunoglobulins:

Anti apolipoprotein B IgG can be obtained from various sources or produced as described in examples 2 and 3. They can be refrigerated at 2 to 8° C. for use in the week, or frozen at −20° C. for use up to months. The immunoglobulins contain sodium azoture.

Standard:

Apo CIII standard is a human serum pool calibrated with electroimmunodiffusion assay and tested for HIV and hepatitis viruses, handled according to the usual precautions in order to prevent contamination. Standard apo CIII level is 2.5 mg/dl. For constructing the calibration curve, standard is diluted as indicated in Table 2.

TABLE 2

| Points | Diluant 2 (μl) | Standard (μl) | Concentration (μg/ml) |
|---|---|---|---|
| 1 | 175 | 25 | 0.3125 |
| 2 | 165 | 35 | 0.4375 |
| 3 | 150 | 50 | 0.6250 |
| 4 | 140 | 60 | 0.7500 |
| 5 | 130 | 70 | 0.8750 |
| 6 | 120 | 80 | 1.000 |
| 7 | 100 | 100 | 1.250 |
| 8 | 60 | 140 | 1.750 |
| 9 | 0 | 200 | 2.500 |

Sample Preparation:

Fresh or frozen (−80° C.) samples are recommended for analysis. Sera are collected according to established procedures in clinical laboratory testing. If needed, samples can be kept frozen for longer storage periods; frozen samples are stable for up to one year. Prior to use, the samples are diluted 3 fold in the diluent 1.

Preparation of Samples without Apo B Particles:

In a test tube, add in the following order: 40 μl anti apo B, 40 μl of serum and 40 μl of diluant 1 of Beckman. Vortex and incubate the mixture 10 minutes at room temperature, and centrifuge at 3500 rpm for 10 minutes. The supernatant is collected for analysis. The final concentration of the samples without apoB is corrected because the 3 fold dilution of the supernatant.

Protocol:

Program a user-defined reagent with the parameters listed below according to the IMMAGE Immunochemistry System Operations Manual.

Transfer antibody reagent to compartment A of a new User Defined Cartridge.

Transfer buffer 1 to compartment B of the cartridge.

Enter the value from the standard (actual standard apo CIII value is 2.5 mg/dl) in parameter table according to the dilution scheme shown in table 2.

Use Diluent 2 as the sample diluent.

Summary:

| | | | |
|---|---|---|---|
| Chem Name | Apo CIII | Units | mg/dl |
| Lot Number | See Cartridge | Protocol | Non-Competitive |
| | | | Nephelometric |
| Reagent Serial | See Cartridge | Reagent Expiration | To be defined by |
| | | | the user |
| Sample or | 20 μl | Gain | 3 |
| Dilution | | | |
| Volume | | | |
| Reagent Buffer | 0 μl | Cal Dilution | 1/5 |
| Volume | | | |
| Compartment | 20 μl | Sample Dilution | 1/20* |
| Volume | | | 1/5** |
| Compartment | 200 μl | Reaction time | 2 minutes |
| Volume | | | |

*To be configured after calibration approval.
**To be configured for apo CIII Lp non B measurement.

Results:

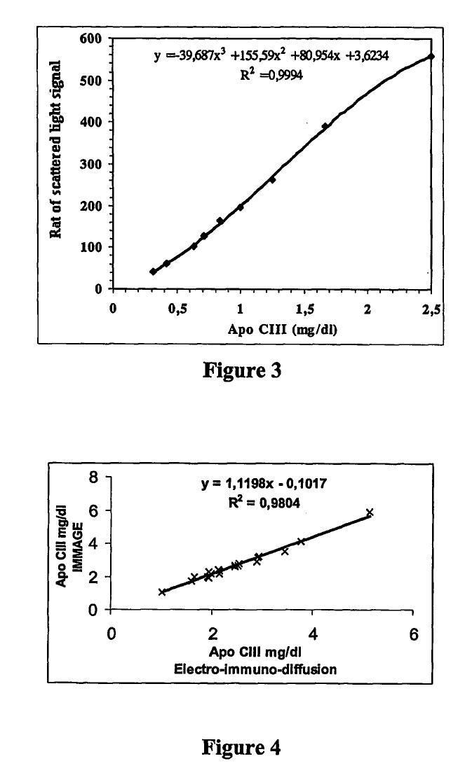

The results are presented on FIG. 3. They show a very narrow coefficient of variation with a working range of 0.3-2.5 μg/ml. Reference Values obtained are: Total Apo CIII: 1.6 to 4.5 mg/dl; Apo CIII Lp non B: 0.5 to 3.5 mg/dl; Apo CIII LpB: <2.3 mg/dl.

7. Comparison of the Apo CIII Nephelometric Assay and Apo CIII Electroimmunodiffusion Assay.

The characteristicts and performance of the nephelometric assay of this invention were compared to those obtained with an electroimmunodiffusion assay (32). The results are reported in Table 3 below and illustrate the advantages of the nephelometric assay of this invention.

TABLE 3

| | Nephelometric assay | | Electroimmunodiffusion | |
|---|---|---|---|---|
| | Total | Apo CIII | Total | Apo CIII |
| | apo CIII | Lp non B | apo CIII | Lp non B |
| CV (%) | 2 | 4.2 | 9.7 | 7.7 |
| Recovry (%) | 101 | 105 | 103 | ND* |

| | | |
|---|---|---|
| Sensitivity | 0.06 | 0.09 |
| (mg/dl) | | |
| Delay (for 20 | 90 minutes | 1 day |
| samples) | | |

*Non Determined

8. Correlation Study

20 serum samples analysed on the electro-immuno-difusion using SEBIA kit of Lp CIII were subsequently analysed using the IMMAGE method. The results are shown on FIG. 4 and demonstrate the high correlation and accuracy of the nephelometric assay using antibodies of this invention.

9. Other Advantages of the Invention

Other advantages and uses of the synthetic apo CIII polypeptides of this invention include:

use as a standard for the calibration of all the apo CIII assays (ELISA, RIA, electroimmunodifusion, etc.)

use in the investigation of the metabolic pathways of lipoproteins, like the inhibition of LpL activity (this enzyme is involved in the lipolysis of triglyceride-rich lipoprotein), or the inhibition of the uptake of apo B-containing lipoproteins by the LDL receptor.

Other advantages and uses of the antibodies of this invention include:

use in all the immuno assays to quantify apo CIII.

Use in the detection of apo CIII (Immunoblot, dot blot, immunohistochemistry and immunocytochemistry)

Use in immuno-affinity and immunoprecipitation methods to purify the protein.

### References

1. Hoddis, H. N., and Mack W. J., Triglyceride-rich lipoproteins and the progression of coronary artery disease. Curr. Opin. Lipidol., 1995. 6: p. 209-214.

2. Brewer, H. B., Shulman R., Herbert P., Roman R. and Werrly K., The complete amino acids sequence of alanine apolipoprotein from plasma very low density lipoprteins. J. Biol. Chem., 1974. 249: p. 4975-4984.

3. Lenich, C., Brecher P., Makrides S., Chobanian A. and Zannis V. I., Apolipoprotein gene expression in rabbit: abundance, size and distribution of apolipoprotein mRNA species in different tissues. J. Lip. Res., 1988. 29: p. 755-764.

4. Ito, Y., Breslow J. L., Chait B. T., Apolipoprotein C-III0 lacks carbohydrate residues: use of mass spectrometry to study apolipoprotein structure. J Lipid Res, 1989. 30: p. 1781-1787.

5. Assman, G., and Ulenbruck G., Characterization of oligosaccharide side chain of apolipoprotein CIII from human VLDL plasma. Biochimica and Biophysica Acta, 1989. 541: p. 234-240.

6. Pullinger, C. R. Malloy M. J., Shahidi A. K., Ghassemzadeh M. Duchateau P., Villagomez J., Allaart J. and Kane J. P., A novel apolipoprotein C-III variant, apoC-III(Gln38-->Lys), associated with moderate hypertriglyceridemia in a large kindred of Mexican origin. J Lipid Res, 1997. 38: p. 1833-1840.

7. von Eckardstein, A., Holz H., Sandkamp M., Weng W., Funke H., Assmann G., Identification of an apolipoprotein C-III variant in a family with hyperalphalipoproteinemia. J Clin Invest, 1991. 87: p. 1724-1731.

8. Roghani, A., and Zannis V. I., Mutagenesis of the glycosylation site of human Apo CIII. O-linked glycosylation is not required for Apo CIII secretion and lipid binding. J Biol Chem, 1988. 263: p. 17925-17932.

9. Schonfeld, G., George P. K., Miller J., Reilly P. and Witztum J., Apolipoprotein C-II and C-III levels in hyperlipoproteinemia. Metabolism, 1979. 28(10): p. 1001-10.

10. Kashyap, M. L., Srivastava L. S., Hynd B. A., Gartside P. S. and Perisutti G., Quantitation of human apolipoprotein C-III and its subspecie by radioimmunoassay and analytical isoelectric focusing: abnormal plasma triglyceride-rich lipoprotein apolipoprotein C-III subspecie concentrations in hypertriglyceridemia. J Lipid Res, 1981. 22: p. 800-810.

11. Shelburne, F., Hanks J., Meyers W. and Quardfordt S. A., Effect of apolipoproteins on hepatic uptake of triglyceride emulsions in the rat. J. Clin. Inves., 1980. 65: p. 652-658.

12. Windler, E., and Havel R. J., Inhinbitory effect of C apolipoproteins from rats and human on the uptake of triglyceride-rich lipoproteins and their remnant by the perfused rat liver. J. Lip. Res., 1985. 26: p. 556-563.

13. Brown, V., and Bakinsky M. L., Inhition of lipoprotein lipase by an apolipoprotein of human very low densty lipoproteins. Biochim. Biophs. Acta., 1972. 46: p. 375-382.

14. Krauss, R. M., Herbert P. N., Levy R. I. and Fredrickson D. S., Further observations on the activation and inhibition of lipoprotein lipase by apolipoproteins. Circ. Res., 1973. 33: p. 403-411.

15. Wang, C. S., Mc Conathy W. J., Kloer H. U. and Alaupovic P., Modulation of lipoprotein lipase activity. The effect of apolipoprotein CIII. J. Clin. Inves., 1985. 75: p. 384-390.

16. Mc Conathy, W. J., Gesquiere J. C., Mass H., Tartar A., Fruchart J. C. and Wang C. S., Inhibition of lipoprotein lipase activity by synthetic peptides of apo CIII. J. Lip. Res., 1972. 33: p. 995-1003.

17. Kinnemen, P. K. J., and Enholm C., Effect of serum and C apoproteins from YLDL on post-heparin plasma hepatic activity. FEBS, 1976. 65: p. 354-357.

18. Blankenhorm, D. H., Alaupovic P, Wickham E., Chin H. P. and Azen S. P., Prediction of angiographic change in native human coronary bypass grafts lipid and non lipid factors. Circulation., 1990. 81: p. 470-476.

19. Hoddis, H. N., Mack W. J., Azen S. P., Alaupovic P., Pogogla J. M., Labree L., Hemphill L. C., Kramsch D. M. and Blackerhorm D. H., Triglyceride and cholesterol rich lipoproteins have differential effect on mild/moderate and severe lesion progression as assessed by quantitative coronary angiography in controlled trial lovastatin. Circulation., 1994. 90: p. 42-49.

20. Koren, E., Corder C., Mueller G., Centurion H., Hallum G., Fesmire J., Mc Conathy W. J. and Alaupovic P., Triglyceride-rich lipoprotein lipoparticles correlate with severity of coronary disease. Atherosclerosis, 1996. 122. p. 105-115.

21. Aalto-Setälä, K., Fisher E. A., Chen X., Chajek-Shaul T., Hayek T., Zechner R., Walsh A., Ramakrishnan R., Ginsberg H. N. and Breslow J. L., Mechanism of hypertriglyceridemia in human apolipoprotein (apo) CIII transgenic mice. Diminished very low density lipoprotein fractional catabolic rate associated with increased apo CIII and reduced apo E on the particles. J Clin Invest, 1992. 90: p. 1889-1900.

22. Harrold, H. J., Van Barlinger J., Harmen de J., Erklens W. D. and Tjerk W. A de Bruin., Lipoprotein lipase enhanced binding of human triglyceride rich lipoproteins to heparan sulfate: Modulation by apolipoprotein E and apolipoprotein C. J. Lip. Res., 1996. 37: p. 754-760.

23. Yang, C. Y., Gu Z. W., Valentinova N., Pownall H. J., Lee B., Yang M., Xie Y. H., Guyton J. R., Vlasik T. N., Fruchart J. C., and Gotto A. M., Human very low density lipoprotein structure: interaction of the C apolipoproteins with apolipoprotein B-100. J Lipid Res, 1993. 34: p. 1311-1321.

24. Clavey, V., Lestavel-Delattre S., Copin C., Bard J. M. and Fruchart J. C., Modulation of lipoprotein B binding to the LDL receper by exogenous lipids and apolipoproteins CI, CII, CIII and E. Arth. Thromb. And Vasc. Biol., 1995. 15: p. 963-971.

25. Blankenhorn, D. H., Alaupovic P., Wickham E., Chin H. P. and Azen S. P., Prediction of anglographic change in native human coronary arteries and aortocoronary bypass grafts. Lipid and nonlipid factors. Circulation., 1990. 81: p. 470-476.

26. Holmquist, L., Quantitation of human serum very low density apolipoproteins C-I, C-II, C-III and E by enzyme immunoassay. J Immunol Methods, 1980. 34(3): p. 243-51.

27. Curry, M. D., McConathy W. J., Fesmire J. D. and Alaupovic P., Quantitative determination of human apolipoprotein C-III by electroimmunoassay. Biochim Biophys Acta, 1980. 617(3): p. 503-13.

28. Merrifield, R. B., Solide phase peptide synthesis. The synthesis of a tetrapeptide. J. Am. Chem. Soc, 1963. 85: p. 2149-2154.

29. Vaitukaitis, J., et al., A method for producing specific antisera with small doses of immunogen. J Clin Endocrinol Metab, 1971. 33(6): p. 988-91.

30. Bassiri, R. M., Dvorak J. and Utiger R. D., Thyrotrpin-releasing hormone, in Methods of hormone radioimmunoassay, B. M. In: Jaffe, and Behrnan H. R. (Eds), Editor. 1979, New York Academic Press: New York. p. p: 46.

31. Ritchie, R. F., and J. Stevens, Qualifications for acceptable anti-serum performence in the automated immunprecipitatin system: A brief review of commercially available reagents, in in advances in Automated Analysis, Technicon Symposia. 1972, Mediad Inc: Tary-town, N.Y. p. 9-14.

32. Luc, G., Fievet C., Arveiler D., Evans A. E., Bard J. M., Combien F, Fruchart J. C. and Ducimetiere P., Apolipoproteins CIII and E in apo B and non apo B containing lipoproteins in two populations at contrasting risk for myocardial infarction: the ECTIM study. J. Lipid. Res., 1996. 37: p. 508-517.

1. A method of detecting apo CIII or apo CIII in apo B and non apo B containing lipoparticles in a biological sample, comprising (i) contacting the sample with an antibody raised by immunization with a substantially pure, synthetic polypeptide comprising amino acid sequence SEQ ID NO: 1, or with a fragment or derivative of said antibody having substantially the same antigenic specificity and (ii) detecting the presence of antibody-antigen immune complexes.

2. The method of claim 1, wherein the presence of antibody-antigen immune complexes is determined by ELISA, RIA, sandwich immuno-assay or direct immunoassay.

3. The method of claim 1, wherein the presence of antibody-antigen immune complexes is determined by nephelometric assay.

4. The method of claim 1, wherein the antibody is obtained by injecting a substantially pure, synthetic polypeptide comprising amino acid sequence SEQ ID NO: 1 to an non-human animal and collecting the antibodies or antibody-producing cells.

5. The method of any one of claims 1 to 4, wherein the antibody is a polyclonal antibody.

6. The method of any one of claims 1 to 4, wherein the antibody is a monoclonal antibody.

7. A method of detecting apo CIII or apo CIII-containing lipoparticles in a biological sample, comprising (i) contacting the sample with an antibody that binds a substantially pure, synthetic polypeptide comprising amino acid sequence SEQ ID NO: 1, or with a fragment or derivative of said antibody having substantially the same antigenic specificity and (ii) assessing the formation of apo CIII-antibody immune complexes by nephelometric assay.

8. The method of claim 7, wherein the antibody is a polyclonal antibody obtained by injecting a substantially pure, synthetic polypeptide comprising amino acid sequence SEQ ID NO: 1 to an non-human animal and collecting the antibodies.

9. The method of any one of claims 1 to 8, wherein the biological sample is a blood sample or a serum sample.

10. A method of detecting predisposition or individuals at risk of developing lipid-metabolism disorders, comprising detecting in vitro apo CIII or apo CIII in apo B and non apo B containing lipoparticles in a sample from a subject with an antibody that binds a substantially pure, synthetic polypeptide comprising amino acid sequence SEQ ID NO: 1, or with a fragment or derivative of said antibody having substantially the same antigenic specificity.

11. A method of monitoring the efficacy of a lipid-metabolism-related disorder treatment in a subject comprising detecting total apo CIII and apo CIII in apo B and non apo B containing lipoparticles levels in vitro in a sample from said subject using an antibody that binds a substantially pure, synthetic polypeptide comprising amino acid sequence SEQ ID NO: 1, or with a fragment or derivative of said antibody having substantially the same antigenic specificity.

12. Use of an antibody that binds a substantially pure, synthetic polypeptide comprising amino acid sequence SEQ ID NO: 1, or of a fragment or derivative of said antibody having substantially the same antigenic specificity, to screen in vitro compounds or diets that modulate apo CIII apo B and non apo B containing lipoparticles concentration in serum.

13. A kit comprising an antibody that binds a substantially pure, synthetic polypeptide comprising amino acid sequence SEQ ID NO: 1, or a fragment or derivative of said antibody having substantially the same antigenic specificity, and a reagent for performing an antigen-antibody immune reaction.
